(19) United States
(12) Patent Application Publication
(10) Pub. No.: US 2018/0120455 A1
(43) Pub. Date: May 3, 2018

(54) DETECTOR MODULE FOR AN X-RAY DETECTOR, X-RAY DETECTOR, AND X-RAY DEVICE

(71) Applicant: Siemens Healthcare GmbH, Erlangen (DE)

(72) Inventor: Alfons EISMANN, Pinzberg (DE)

(73) Assignee: Siemens Healthcare GmbH, Erlangen (DE)

(21) Appl. No.: 15/729,786

(22) Filed: Oct. 11, 2017

(30) Foreign Application Priority Data
Oct. 27, 2016 (DE) 102016221209.5

(51) Int. Cl. G01T 1/24; A61B 6/00; A61B 6/03; G01T 1/16; G01T 1/29; G01T 1/172

(52) U.S. Cl. CPC G01T 1/243; A61B 6/4441; A61B 6/037; A61B 6/425; A61B 6/547; G01T 1/1603; G01T 1/2985; G01T 1/172; A61B 6/4266

(57) ABSTRACT

A detector module is for an X-ray detector. An embodiment of the detector module includes an X-ray-sensitive layer; readout electronics assigned to the X-ray-sensitive layer; and a first interface. In an embodiment, the first interface is embodied both for power transmission to the readout electronics and for data transmission from the readout electronics. An X-ray detector of an embodiment includes a number of corresponding detector modules and an X-ray device includes such an X-ray detector.

### PRIORITY STATEMENT

The present application hereby claims priority under 35 U.S.C. § 119 to German patent application number DE 102016221209.5 filed Oct. 27, 2016, the entire contents of which are hereby incorporated herein by reference.

### FIELD

At least one embodiment of the invention generally relates to a detector module for an X-ray detector. At least one embodiment of the invention also generally relates to an X-ray detector for recording an image of an object radiographed by X-rays. At least one embodiment of the invention furthermore generally relates to an X-ray device with an X-ray detector.

### BACKGROUND

An X-ray detector generally forms part of an X-ray device and is typically used in imaging applications, such as, for example, for computed tomography recordings in medical imaging in order to produce a three-dimensional image of a region of interest of a patient. A conventional X-ray device typically comprises an X-ray source and the X-ray detector.

For the recording of X-ray images, an object to be scanned is positioned between the X-ray source and the X-ray detector. In the case of an X-ray device for medical applications, the object to be scanned is usually a patient. The X-ray source then projects X-rays onto the object to be scanned. The X-rays penetrate the object to be scanned and are attenuated thereby to different degrees (depending upon the density of the object to be scanned). The X-ray detector is used to detect the intensities of the X-rays (transmitted through the object and hence attenuated).

In X-ray detectors, in principle, a distinction is made between indirect-conversion X-ray detectors and direct-conversion X-ray detectors. In order to detect the transmitted X-rays, an indirect-conversion X-ray detector has an X-ray sensitive (sensor) layer made of a scintillator material, which converts the incident X-rays into visible light. This visible light is then converted via a photodiode into an electrical signal. In the case of a direct-conversion X-ray detector, the sensor layer is made of a semiconductor material such as, for example, cadmium telluride in which incident X-rays are directly converted into an electrical signal. The respective electrical signals are detected, collected and optionally buffered, amplified and converted into digital signals in readout electronics (also called backend electronics) assigned in each case to the detector module.

Against the background of simple and economical manufacturing of a large-area X-ray detector, the entire sensitive detector surface is often composed of smaller, individually-produced detector modules. In such cases, each detector module generally comprises a plurality of detector pixels. This means that each detector module can detect a plurality of signals in a spatially-selective manner and in parallel, which, in a subsequently-formed matrix image composed of the signals of all the detector modules, in each case represent an image point of this matrix image. The detector modules themselves are in turn typically arranged in rows or in an array to form the detector surface.

During the recording of X-ray images, the measurement data (also: measurement signals) generated by the detector modules originating from the detector modules is sent via data-transmission electronics (and in the case of computed tomography usually via a slip ring) to an image computer used for image processing. Herein, the data-transmission electronics collect the data from the detector modules, process it and retransmit it in logged and bundled form. Transmission is either directly to the image computer or, for example in the case of a very large number of detector modules with a plurality of (decentralized) distributed electronic printed circuit boards, to a central data interface, which in turn takes over transmission of the detector data to the image computer.

Regardless of the transmission route, the respective detector module, in particular the backend electronics thereof, requires means both for data transmission away from the backend electronics and for power transmission to the backend electronics. These means are usually formed from suitably embodied, physically different, interfaces. Herein, power transmission is usually performed with the aid of comparatively thick (copper) cables, while data transmission takes place using comparatively thin copper wires and/or even glass fibers for correspondingly high data-transmission speeds.

Furthermore, special (data transmission) protocols are frequently also used for internal detector data transmission. In particular, the interfaces of different transmission stages (between the detector module and the electronics and between the electronics and the image computer or the data interface) inside an X-ray device are often also each used with their own protocols, which hence also have to be implemented and verified in different types of control software.

### SUMMARY

At least one embodiment of the invention involves simplifying the assembly of X-ray detectors.

At least one embodiment of the invention is directed to a detector module. At least one embodiment of the invention is directed to an X-ray detector. At least one embodiment of the invention is directed to an X-ray device. Advantageous embodiments and developments of the invention, which are in part inventive in their own right, are set forth in the claims and the following description.

The detector module according to at least one embodiment of the invention is configured and provided for use in an X-ray device. Herein, the detector module comprises an X-ray-sensitive layer, readout electronics assigned to the X-ray-sensitive layer and a first interface. Herein, the first interface is embodied both for power transmission to the readout electronics and for data transmission from the readout electronics (i.e. in particular “away” therefrom, preferably to a higher-ranking data-processing module, which is in particular part of an X-ray detector).

The X-ray device according to an embodiment of the invention comprises the X-ray detector of the above-described type. The X-ray device also preferably comprises an (X-ray) radiation source for radiography of an object to be examined, such as, for example, a patient.

### BRIEF DESCRIPTION OF THE DRAWINGS

The following shows an example embodiment of the invention with reference to a drawing, which shows:

FIG. 1 shows a schematic and sectional view of an X-ray device with an X-ray detector comprising a plurality of detector modules.

Parts corresponding to one another are always given the same reference numbers in all figures.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. The present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “exemplary” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or porcessors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (procesor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

The detector module according to at least one embodiment of the invention is configured and provided for use in an X-ray device. Herein, the detector module comprises an X-ray-sensitive layer, readout electronics assigned to the X-ray-sensitive layer and a first interface. Herein, the first interface is embodied both for power transmission to the readout electronics and for data transmission from the readout electronics (i.e. in particular “away” therefrom, preferably to a higher-ranking data-processing module, which is in particular part of an X-ray detector).

This means that the power supply for a detector module (or the respective components thereof) required for recording an image and the data obtained during the recording of an image are transmitted via a common interface (preferably with a high transmission rate). In particular, only one single interface is provided for each detector module. As a result, it is advantageously possible to save on separate plug connectors with the respective terminal contacts required for data transmission and power transmission inside the detector module.

Hence, the integration of power transmission and data transmission in only one interface according to at least one embodiment of the invention advantageously greatly simplifies assembly and installation of detector modules in an X-ray detector. Herein, preferably (in particular when installed as intended in the X-ray detector) one common line is also used for power and data transmission to or from the detector module (i.e. in particular only one single cable) so that in particular it is also possible to reduce what would otherwise be a comparably high cabling requirement in view of separate cables for the data transmission and power supply. In this case, hence, the power supply is preferably provided via a data network, which is used to transmit the measurement signals inside the X-ray detector and/or the X-ray device comprising the X-ray detector.

Herein, it is advantageously also possible to use standardized, commercially available interfaces permitting both data transmission and power transmission. Preferably, such a standardized interface is also actually used and hence replaces the previously used separate and complicated data and power transmission interfaces.

Here and hereinafter, a detector module designates a unit of an X-ray detector. A detector module has an X-ray-sensitive layer—i.e. sensor layer that is sensitive to X-rays—for converting X-rays directly into an electrical signal (in the case of a direct-conversion detector) or into visible light (in the case of an indirect-conversion detector). In the latter case, a photodiode is arranged downstream of the X-ray-sensitive layer for the conversion of the visible light into an electrical signal. In both cases, the detector module preferably further also comprises a capacitor (i.e. in particular a condenser) for buffering the electrical signal. Expediently, the detector module also comprises a plurality of detector pixels (and hence in particular also a plurality of photodiodes and/or condensers).

The detector module also comprises the readout electronics assigned to the X-ray-sensitive layer. The X-ray-sensitive layer is located upstream of the readout electronics in particular in the direction of incidence of the X-rays. In the case of a direct-conversion detector, the X-ray-sensitive layer is in direct electrical contact with the readout electronics. Herein, the readout electronics preferably comprise an ASIC, which is used, for example, to amplify, filter, smooth, or the like, the measurement signals.

Here and hereinafter, a higher-ranking data-processing module, also called data handling front end electronics (DHFEE), should be understood to be an electronic system on which in particular data (measurement signals) received from a plurality of detector modules during the recording of an image (via an X-ray detector or in a corresponding X-ray device) is transmitted. The data-processing module is in particular used for data preparation, encoding the prepared data and bundling thereof for further outputting.

The outputting of the data originating from the data-processing module is then preferably performed on a correspondingly higher-ranking control and evaluation unit, which is expediently part of an X-ray device used to record X-ray images. The control and evaluation unit expediently comprises a microcontroller and/or a FPGA (field programmable gate array). It is further expedient for the control and evaluation unit to comprise a memory in which image calculation functions can be implemented in an executable way.

The X-ray detector according to an embodiment of the invention comprises a number of modules of the type described above arranged adjacent to one another and forming a detector surface. I.e. the respective detector module comprises the X-ray-sensitive layer, the readout electronics assigned to the X-ray-sensitive layer and the first interface. Herein, the respective first interface is embodied both for power transmission to the corresponding readout electronics and for data transmission from the readout electronics (preferably to the higher-ranking data-processing module).

Hence, the X-ray detector according to an embodiment of the invention is used to record an image of an object radiographed by X-rays. In particular, the X-ray detector according to an embodiment of the invention is configured and provided for use in a (medical or industrial) X-ray device, preferably a computed tomography scanner. Alternatively, it is also possible in the context of an embodiment of the invention to use the X-ray detector in another device comprising a large detector surface with a plurality of elements, for example a C-arm X-ray device or an electron microscope with X-ray analysis.

In one example embodiment, for each detector module, the X-ray detector uses a standardized interface via which both data and power can be transmitted. Such interfaces enable the replacement of the separate interfaces commonly used to date for data transmission and power transmission by one common (single) interface. Herein, as described above, preferably a common line, in particular a common cable for power and data transmission is used for each (first) interface. In particular, in the case of large-surface X-ray detectors with a plurality of detector modules, this advantageously simplifies assembly due to the lower number of components required (cables, plugs, etc.).

In a further example embodiment, the X-ray detector comprises the above-described data-processing module. Herein, the respective detector module is expediently connected in a signal-transmitting manner via the first interface (and preferably in each case via a common line) to the data-processing module. Hence, the data transmission from the detector module to the data-processing module in particular takes place directly. In addition, the respective detector module is supplied with power via the first interface from the data-processing module. Hence, in the present case, the data-processing module is also expediently configured to provide power, in particular to convert a voltage value of a supply voltage provided to the data-processing module to an operating voltage value necessary to operate the detector module. Preferably, the data-processing module in each case comprises a first “counter-interface” corresponding to the respective first interface of the detector module to which a corresponding line (in particular a cable) leading to the respective detector element is connected.

In one expedient embodiment, the first interface is integrated in the respectively assigned detector module, in particular in the readout electronics thereof. Herein, integration of the interface in particular reduces the installation space occupied by the detector module. Preferably, herein, the interface is positioned on a circuit board of the readout electronics of the respective detector module (for example in the form of a corresponding plug for connecting a line or cable).

In a further expedient embodiment, the data-processing module (preferably in a direction facing away from the respective detector module in the data flow direction) comprises a second interface for transmission and for the power supply. Herein, this second interface is in particular used for electrical (also: signal-transmitting) connection to a higher-ranking unit, in particular a control and evaluation unit, which is preferably assigned to the X-ray device comprising the X-ray detector. Herein, similarly to first interface, the second interface used is preferably also a common interface embodied both for power transmission to the data-processing module (preferably from the control and evaluation unit) and for data transmission (away) from the data-processing module to the control and evaluation unit. Hence, the power supply for the data-processing module is provided, not separately, but jointly with the data transmission (in particular counter to the data flow direction) via the common interface and preferably via one and the same line element (in particular a common cable). Hence, this advantageously enables the assembly and cabling costs to be further reduced.

Like the first interface in the detector module, the second interface is preferably integrated in the data-processing module. Hence, owing to this integration of the interface, here once again, the assembly of a corresponding X-ray detector can require less installation space—compared to X-ray detectors used at present.

In one conceivable embodiment in the context of the invention, the X-ray detector comprises a plurality of data-processing modules to each of which a group of a plurality of detector modules is connected. Such “decentralization” enables the cabling of in particular an elongated X-ray detector, i.e. an X-ray detector with multiple detector modules arranged in line next to one another but only relatively few detector modules arranged in series one behind the other, to be simplified.

In a further advantageous embodiment of the invention, the X-ray detector comprises a data-control unit connected (in particular in a signal-transmitting manner) downstream of the or each data-processing module (hence, in the context of the X-ray device, preferably inserted between the data-processing module and the control and evaluation unit). The data-control unit, also called a data transmission controller (DTC), is in particular used to transmit the data prepared in the data-processing module to the control and evaluation unit. Optionally, the data-control unit is also used to encode, compress, or the like, the data in accordance with a protocol used for data transmission to the control and evaluation unit.

In particular, in the case of a plurality of data-processing modules, in one expedient embodiment, the data-control unit is embodied as a separate component and connected as such connect to the or the respective data-processing module. With an embodiment of this kind, the data-processing module is expediently connected to the data-control unit via the second interface (and expediently comprises a second “counter-interface” corresponding to the second interface). The data-control unit then preferably comprises a third interface for connection to the control and evaluation unit of the X-ray device. This third interface is also preferably embodied for power transmission to the data-control unit (preferably from the control and evaluation unit) and for data transmission from the data-control unit (in particular to the control and evaluation unit).

In one alternative advantageous embodiment, the data-control unit is part of the data-processing module. In other words, the data-control unit is preferably integrated in the or the respective data-processing module. Accordingly, only two interfaces (or optionally interface pairs consisting of a first interface and a first counter-interface and a second interface and a second counter-interface) are required, in order, when the X-ray detector is installed as intended in the X-ray device, to ensure data transmission from the respective detector module of the X-ray detector to the control and evaluation unit of the X-ray device and the power supply to the components of the X-ray detector.

In a further advantageous embodiment, the first interface and/or the second interface (and optionally the third interface and/or the corresponding counter-interfaces) are embodied as Power over Ethernet interfaces. The use of in particular standardized Power over Ethernet (“POE”) technology advantageously enables the design of the X-ray detector to be further simplified since it is preferably also possible to use standardized data transmission protocols. Hence, there is no need for an “extra” transmission protocol with interface-specific implementation.

A Power over Ethernet interface also advantageously enables up to 25.5W (48V) power (or voltage) to be transmitted for each POE interface. Future Power over Ethernet-interfaces will enable power transmission of up to 100W. In addition, data rates of up to 1 Gigabit/s (“Gigabit Ethernet”) are possible. These standardized Gigabit-Ethernet data transmission technologies including the integrated power interface (with powers of up to 25.5W at 48V) replace the previously used separate and elaborately developed data interfaces inside the X-ray detector and preferably from the X-ray detector to the higher-ranking components of the X-ray device, in particular the CT-detector. This means a significant simplification in the development of detector-internal interfaces. Furthermore, the 48V technology of the Power over Ethernets enable power to be transmitted at lower currents inside the X-ray detector. Thus, the power losses inside the cable are reduced.

The X-ray device according to an embodiment of the invention comprises the X-ray detector of the above-described type. The X-ray device also preferably comprises an (X-ray) radiation source for radiography of an object to be examined, such as, for example, a patient.

In one example embodiment, the X-ray device also comprises the above-described control and evaluation unit, which is, in particular connected to the data-processing module or optionally to the data-control unit via the second or third interface. Herein, the control and evaluation unit is expediently used both as an operator device for controlling the X-ray detector for recording images and for calculating and displaying the X-ray images. Herein, in the context of the invention, the control and evaluation unit can be embodied as a non-programmable electronic circuit and also, for example, in a controller of the X-ray device. However, preferably, the control and evaluation unit, or at least the core thereof, is formed by a microcontroller and an assigned memory in which the functions of image calculation and image display are implemented in the form of a software module. Preferably, the control and evaluation unit is a computing unit (for example an industrial PC, a workstation or the like) assigned to the X-ray device.

In principle, in the context of embodiments of the invention, the X-ray device can be such a device with a large-area, flat and, particularly during operation, immobile X-ray detector. However, preferably, the X-ray device is a computed tomography scanner wherein, with a length of approximately 1 m, the X-ray detector thereof is regularly elongate compared to its width. Accordingly, the X-ray detector is preferably such a detector for a computed tomography scanner.

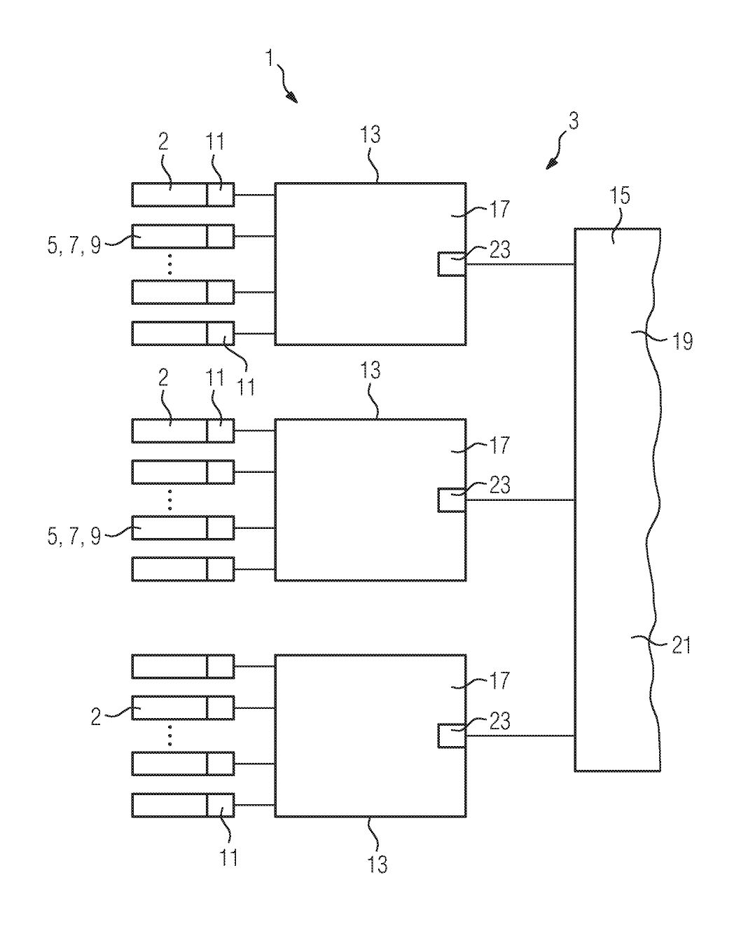

FIG. 1 is a schematic view of an X-ray device 1 comprising an X-ray detector 3 comprising a plurality of detector modules 2. Each of the detector modules 2 of the X-ray detector 3 comprises an X-ray-sensitive layer 5 and readout electronics 7 assigned to the X-ray-sensitive layer 5.

The detector modules 2 are arranged adjacent to one another. Each X-ray-sensitive layer is part of a detector surface 9. Therefore, the X-ray-sensitive layers 5 jointly form the detector surface 9. The X-ray-sensitive layers 5, the readout electronics 7 and the detector surface 9 are only indicated here and, for reasons of clarity, only partially depicted.

A first interface 11 is integrated in each detector module 2. This interface 11 connects the respective detector module 2 to a higher-ranking data-processing module 13 of the X-ray detector 3. In the present case, three data-processing modules 13 are shown, each of which are connected to a plurality of detector modules 2. The respective data-processing module 13 in each case also comprises, in a way that is not depicted in further detail, a counter-interface corresponding to the respective interface 11. Inside the data-processing module or modules 13, the measurement signals (also measurement data) generated within the context of the image recording by the detector modules 2 are prepared, encoded and bundled.

The interface 11 is a standardized Power over Ethernet interface by which the energy or power supply to the detector modules 2 required for the image recording and the data transmission originating from the detector modules 2 to the data-processing module 13 takes place integrally. Hence, only one interface 11 for data and power transmission is provided for each detector module 2 so that there is no need for separate plugs and cables inside or on the detector modules 2, such as those it was necessary to date to provide separately for power transmission.

A control and evaluation unit 15 assigned to the X-ray device 1 is connected downstream of the X-ray detector 3, specifically the data-processing modules 13. Also integrated in the or the respective data-processing module 13 is a data-control unit 17, which is used to transmit the data prepared in the respective data-processing module 13 to the control and evaluation unit 15.

The control and evaluation unit 15 controls the X-ray detector 3 in the context of the X-ray image recording and is simultaneously used for image calculation and image display. To this end, the control and evaluation unit 15 comprises a microcontroller 19 and an assigned memory 21 in which image calculation functions are implemented in an executable way.

The data-processing modules 13 are each connected via a second interface 23 to the control and evaluation unit 15. The respective second interfaces 23 are also embodied as Power over Ethernet interfaces and are each integrated in the corresponding data-processing module 13. Hence, here once again, transmission of the prepared data originating from the data-processing modules 13 and power transmission to the data-processing modules 13 take place via a common interface 23.

The subject matter of the invention is not restricted to the above-described example embodiment. Instead, further embodiments of the invention can be derived by the person skilled in the art from the above description.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A detector module for an X-ray detector, comprising:
an X-ray-sensitive layer;
readout electronics assigned to the X-ray-sensitive layer; and
a first interface, embodied both for power transmission to the readout electronics and for data transmission from the readout electronics.

2. An X-ray detector for recording an image of an object radiographed by X-rays, comprising:
a number of detector modules, arranged adjacent to one another to form a detector surface, wherein each respective detector module of the number of detector modules includes
an X-ray-sensitive layer,
readout electronics assigned to the X-ray-sensitive layer, and
a first interface, each respective first interface being embodied both for power transmission to a respective readout electronics and for data transmission from the respective readout electronics to a relatively higher-ranking data-processing module.

3. The X-ray detector of claim 2, further comprising a data-processing module, wherein a respective one of the number of detector modules is connected to the data-processing module via the respective first interface.

4. The X-ray detector of claim 2, wherein each of the respective first interfaces is integrated in the each of the respective detector module.

5. The X-ray detector of claim 4, wherein the data-processing module comprises a second interface, configured both for power transmission to the data-processing module and for data transmission from the data-processing module.

6. The X-ray detector of claim 3, further comprising a data-control unit, connected downstream of the data-processing module.

7. The X-ray detector of claim 6, wherein the data-control unit is part of the data-processing module.

8. The X-ray detector of claim 2, wherein each respective first interface is embodied as a Power over Ethernet interface.

9. An X-ray device, comprising the X-ray detector of claim 2.

10. The X-ray device of claim 9, wherein a control and evaluation unit is connected downstream of the X-ray detector.

11. The X-ray detector of claim 3, wherein each of the respective first interfaces is integrated in the each of the respective detector module.

12. The X-ray detector of claim 11, wherein the data-processing module comprises a second interface, configured both for power transmission to the data-processing module and for data transmission from the data-processing module.

13. The X-ray detector of claim 5, wherein at least one of the respective first interface and the respective second interface is embodied as a Power over Ethernet interface.

14. An X-ray device, comprising the X-ray detector of claim 3.

15. The X-ray device of claim 14, wherein a control and evaluation unit is connected downstream of the X-ray detector.

16. An X-ray detector for recording an image of an object radiographed by X-rays, comprising:
a plurality of the detector module of claim 1, each of the plurality of detector modules being arranged adjacent to one another to form a detector surface.
